(19) United States
(12) Patent Application Publication
(10) Pub. No.: US 2008/0287556 A1
(43) Pub. Date: Nov. 20, 2008

(54) Process For the Preparation of Fischer-Tropsch Catalysts With a High Mechanical, Thermal and Chemical Stability

(75) Inventors: Giuseppe Bellussi, Piacenza (IT); Luciano Cosimo Carluccio, San Donato Milanese-Milan (IT); Roberto Zennaro, Milano (IT); Gastone Del Piero, Milano (IT)

Correspondence Address: OBLON, SPIVAK, MCCLELLAND MAIER & NEUSTADT, P.C., 1940 DUKE STREET, ALEXANDRIA, VA, 22314, US

(73) Assignees: ENI S.P.A., Rome (IT); INSTITUT FRANCAIS DU PETROLE, Rueil-Malmaison (FR)

(21) Appl. No.: 11/995,834

(22) PCT Filed: Jul. 13, 2006
(86) PCT No.: PCT/EP2006/006949

§ 371 Date: Jul. 29, 2008

(30) Foreign Application Priority Data
Jul. 22, 2005 (IT) MI2005A001410

(51) Int. Cl. C07C 27/06; B01J 31/02

(52) U.S. Cl. 518/715; 502/158

(57) ABSTRACT

The preparation is described of a Fischer-Tropsch catalytic precursor based on cobalt supported on alumina, optionally containing up to 10% by weight of silica, which comprises: a) treatment of the alumina with a silicon compound selected from those having general formula (I) Si(OR)₄₋ₙR′ₙ (I) wherein n ranges from 1 to 3 wherein R′ is selected from primary hydrocarbyl radicals having from 1 to 20 carbon atoms; wherein R is selected from primary hydrocarbyl radicals having from 1 to 6 carbon atoms; b) drying and subsequent calcination of the modified carrier obtained at the end of step (a) thus obtaining a silanized carrier; c) subsequent deposition of cobalt on the silanized carrier obtained at the end of step (b); d) drying and subsequent calcination of the supported cobalt obtained at the end of step (c) thus obtaining the final catalytic precursor; the above final catalytic precursor having a content of SiO₂ deriving from the compound having general formula (I) ranging from 4.5 to 10% by weight.

The present invention relates to a process for obtaining catalysts based on cobalt having a high mechanical, thermal and chemical stability and which can be used for the Fischer-Tropsch reaction, in particular for producing waxes.

Fischer-Tropsch reactions consist in the production of essentially linear and saturated hydrocarbons preferably having at least 5 carbon atoms in the molecule, by the catalytic hydrogenation of CO, optionally diluted with CO₂.

The reaction between CO and H₂ is preferably carried out in a gas-liquid-solid fluidized reactor in which the solid, prevalently consisting of particles of catalyst, is suspended by means of the gaseous stream and the liquid stream. The former prevalently consists of the reagent species, i.e. CO and H₂, whereas the latter consists of the hydrocarbons produced by the Fischer-Tropsch reaction, possibly at least partially recycled, or from material liquid under the process conditions, or relative mixtures.

The gas and optionally recycled liquid are fed from the bottom of the column by means of specific distributors and the gas and liquid flow-rates are such as to guarantee a turbulent flow regime in the column.

In gas-liquid-solid fluidized systems such as that of the Fischer-Tropsch reaction, the fluid flow-rates should be such as to guarantee an almost homogeneous suspension of the solid in the entire reaction volume and facilitate the removal of the heat produced by the exothermic reaction, improving the heat exchange between the reaction area and a suitable exchanger device introduced into the column.

The solid particles, moreover, should have sufficiently large dimensions as to be easily separated from the liquid products, but sufficiently small as to consider the intra-particle diffusion limitations (unitary particle efficiency) negligible and be easily fluidized.

The average diameter of the solid particles used in slurry reactors can vary from 1 to 200 μm, operating with particles having dimensions lower than 10 μm, however, is extremely onerous with respect to the separation of the solid from the liquid products.

From an examination of literature relating to Fischer-Tropsch processes, it is evident that, if, on the one hand, catalysts supported on alumina have excellent catalytic performances in terms of activity and quality of the reaction product (Oukachi et al., Applied catalysis A: General 186, 129-144, 1999; C. H. Bartholomew et al., Journal of Catalysis 85, 78-88, 1984), on the other, there are limits in the stability of the catalytic system due to hydration reactions of the carrier (A. Dolmen Applied Catalysis 186, 169-188, 1999; S. Barradas et al., Studies in Surface Science and Catalysis, 143, 55-65, 2002). The instability of the carrier consequently reduces the operating times of the catalyst in the Fischer-Tropsch reaction.

The problems relating to the stability of the alumina carrier in the Fischer-Tropsch synthesis therefore derive from phenomena of a prevalently chemical nature. During the Fischer-Tropsch reaction, the water produced causes, under suitable temperature and pressure conditions, the hydration of the Al₂O₃, transforming it into boehmite or even pseudoboehmite, with a consequent weakening of the catalyst. It is therefore not only important to obtain excellent performances but also stability with time of the catalyst and in this specific case, the carrier.

One of the various methods for stabilizing alumina consists in the addition of silicon.

The above addition of silicon can be effected with different synthesis procedures:

1. introduction of the silicon directly during the synthesis of the alumina,
2. deposition of the silicon by post-treatment on the pre-formed alumina,
3. deposition of the silicon by post-treatment on the alumina already stabilized according to procedure 1,
4. deposition of the silicon on the catalyst (Al₂O₃+CO₃O₄+SiO₂).

The U.S. Pat. No. 4,013,590 provides an important disclosure, which describes the treatment of alumina (γ, ε, δ, θ-Al₂O₃) with silicon compounds, in particular with alkyl esters of orthosilicic acid, Si(OR)₄. This treatment causes the stabilization of the end-product by decreasing the population of active centres present on the surface of the alumina. Thermal treatment (1200° C.) and hydrothermal treatment (P_{H2O}=15 ata, T=250° C.) do not in fact modify the initial crystalline structure.

EP-A-0180269 describes the preparation of a catalyst in which the carrier is treated with organic compounds of silicon, of the same group cited in the U.S. Pat. No. 4,013,590, in the presence of an organic solvent in order to make the surface of the carrier used less reactive. More specifically, said treatment does not favour the interaction between the carrier and active phase (cobalt), introduced subsequently, which causes the formation of non-active species for the Fischer-Tropsch reaction.

WO-9942214 uses the same organic silicon compounds for making the surface of the alumina less reactive during impregnation in the aqueous phase of the active species (cobalt).

A method for the chemical stabilization (resistance to hydration) of Fischer-Tropsch catalysts has now been found with the use of a particular category of organic compounds of silicon as deposition vectors of silica in alumina. This treatment however does not diminish the catalytic efficiency in the Fischer-Tropsch process.

In accordance with this, the present invention relates to a process for the preparation of a Fischer-Tropsch catalytic precursor based on cobalt supported on alumina, optionally containing up to 10% by weight of silica, which comprises:

a) treatment of alumina with a silicon compound selected from those having general formula (I)

Si(OR)₄₋ₙR′ₙ (I)

wherein n ranges from 1 to 3
wherein R′ is selected from primary hydrocarbyl radicals having from 1 to 20 carbon atoms, R′ is preferably selected from a primary C₁-C₁₀ alkyl radical;
wherein R is selected from primary hydrocarbyl radicals having from 1 to 6 carbon atoms, R is preferably selected from a primary C₁-C₄ alkyl radical;
b) drying and subsequent calcination of the modified carrier obtained at the end of step (a) thus obtaining a silanized carrier;
c) subsequent deposition of cobalt on the silanized carrier obtained at the end of step (b);
d) drying and subsequent calcination of the supported cobalt obtained at the end of step (c) thus obtaining the final catalytic precursor;
the above final catalytic precursor having a content of SiO₂ deriving from the compound having general formula (I) ranging from 4.5 to 10% by weight, preferably from 6% by weight to 7% by weight.

The term “catalytic precursor” is used as, as is well known to experts in the field, the above catalytic precursor must be reduced with hydrogen before being used in the Fischer-Tropsch process.

Typical examples of compounds having general formula (I) are those wherein R and R′, the same or different, are selected from CH₃, CH₂CH₃, (CH₂)₂CH₃, isoC₄H₉, (CH₂)₅CH₃, (CH₂)₇CH₃, (CH₂)₉CH₃, (CH₂)₂CH₃, CHCH₂, C₆H₅.

In the preferred embodiment, R and R′ are selected from CH₃ and CH₂CH₃. Even more preferably, the compound having general formula (I) is methyl-tri-methoxy-silane (R═R′, n=1).

As already mentioned, the starting carrier is selected from alumina optionally containing up to 10% of silica. Any type of alumina can be used (γ, ε, δ, θ-Al₂O₃), preferably γ-alumina. As far as the surface area of the carrier is concerned, this is within the range of 20-300 m²/gr, preferably 50-200 m²/gr (BET).

In a preferred embodiment, step (a) i.e. the deposition of silicon on the carrier, takes place by treatment of the carrier with a solution, preferably ethanolic, of the compound having general formula (I). Solvents different from ethanol can be used, for example, n-hexane, n-heptane, n-octane, toluene, acetonitrile. After a period of stirring, the solvent is eliminated, preferably at reduced pressure, and the solid is dried at a temperature ranging from 100° C. to 160° C. for a time of 2 to 8 hours. This is typically effected in an oven at about 140° C. for 4 hours. Calcination is then carried out (step b) in which the whole organic fraction is burnt. The above calcination takes place at a temperature ranging from 300° C. to 500° C. for a time ranging from 2 to 20 hours in a stream of air. The calcination is typically effected at 400° C. for 16 hours.

Step (c) consists in the deposition of cobalt on the silanized and calcined carrier obtained at the end of step (b). Various techniques can be used for effecting the above step (c), for example gelification, co-gelification, impregnation, precipitation, dry impregnation, coprecipitation. In the preferred embodiment, the cobalt and possible promoters are associated with the carrier by putting the carrier itself in contact with a solution of a compound containing cobalt (or other possible promoters) by impregnation. The cobalt and possible promoters can be optionally co-impregnated on the carrier itself. The compounds of cobalt and possible promoters used in the impregnation can consist of any organic or inorganic metallic compound susceptible to decomposing upon heating in nitrogen, argon, helium or another inert gas, calcination in a gas containing oxygen, or treatment with hydrogen, at high temperatures, to provide the corresponding metal, metal oxide, or mixtures of the metal and metal oxide phases. Compounds of cobalt (and possible promoters) such as nitrate, acetate, acetylacetonate, carbonyl naphthenate and the like, can be used. The quantity of impregnation solution must be sufficient for completely wetting the carrier, normally within a range of about 1 to 20 times the volume of the carrier, in relation to the concentration of metal (or metals) in the impregnation solution. The impregnation treatment can be carried out within a wide range of temperature conditions.

The quantity of cobalt salt to be used is such as to obtain a final catalytic precursor which has a content of CO₃O₄ ranging from 15 to 25% by weight.

The final step (d) is carried out according to the procedure described in step (b).

In the following experimental part, reference is made to a process for the production of catalysts stabilized with respect to hydration phenomena via silanization, various hydration tests simulating extreme conditions for the Fischer-Tropsch process and catalytic tests under Fischer-Tropsch conditions suitable for demonstrating the efficiency of the catalysts obtained after the modifications obtained with the silanization.

The particular efficacy will be shown, of the compounds having general formula (I) with respect to tetraalkoxysilanes, for example TEOS=tetra-ethylorthosilicate.

The following examples are provided for a better understanding of the present invention.

### EXAMPLE 1

### Sample A

Micro spheroidal boehmite is subjected to a first calcination effected at 450° C. for 1 hour, followed by a subsequent calcination at 900° C. for 4 hours in a muffle in a stream of air. An alumina is obtained having the following characteristics:

| | | |
|---|---|---|
| Surface area | 170 | m²g⁻¹ |
| Porous volume | 0.45 | m³g⁻¹ |
| Average particle diameter | 55 | μm |

### EXAMPLE 2

### Sample B

Micro spheroidal boehmite containing 5 wt % of SiO₂, is subjected to a first calcination effected at 450° C. for 1 hour, followed by a subsequent calcination at 1,000° C. for 4 hours in a muffle in a stream of air. An alumina is obtained having the following characteristics:

| | | |
|---|---|---|
| Surface area | 170 | m²g⁻¹ |
| Porous volume | 0.43 | m³g⁻¹ |
| Average particle diameter | 55 | μm |

### EXAMPLE 3

### Sample C 0 wt % SiO₂

50 g of sample A are impregnated in a single step using the wet-imbibition method, with 50 cc of an aqueous solution of cobalt nitrate. This solution is obtained by dissolving 43.55 g of Co(NO₃)₂.6H₂O in such a quantity of water as to reach the above volume. The material is dried in an oven at a temperature of 120° C. for 16 hours in a stream of air and subsequently calcined at a temperature of 400° C. for 4 hours again in a stream of air. The calcined end-product has the following chemical weight composition: 19.4 wt % of CO₃O₄, complement to 100 with Al₂O₃.

The catalyst was subjected to hydrothermal treatment, as described hereunder, and to a catalytic test.

After 120 hours of hydrothermal test, the percentage of boehmite is equal to 20 wt %.

### EXAMPLE 4

### Sample D 4 wt % SiO₂ in Bulk

50 g of sample B are impregnated in a single step using the wet-imbibition method, with 50 cc of an aqueous solution of cobalt nitrate. This solution is obtained by dissolving 43.55 g of Co(NO₃)₂.6H₂O in such a quantity of water as to reach the above volume. The material is dried in an oven at a temperature of 120° C. for 16 hours in a stream of air and subsequently calcined at a temperature of 400° C. for 4 hours again in a stream of air. The calcined end-product has the following chemical weight composition: 19.4 wt % of CO₃O₄, 4.0 wt % of SiO₂, complement to 100 with Al₂O₃.

The catalyst was subjected to hydrothermal treatment, as described hereunder, and to a catalytic test.

After 336 hours of hydrothermal test, the percentage of boehmite is equal to 12 wt %.

### EXAMPLE 5

### Sample E 4 wt % SiO₂ via MTEOS

300 g of sample A, 46.9 g of MTEOS, 300 g of technical ethanol are charged into a rotavapour flask having a volume of 2,000 ml; the reaction mixture is kept for 4 hours at 75° C. under stirring. The solvent and excess of silane are distilled under vacuum. The solid is dried for 4 hours at 140° C. and finally calcined at 400° C. for 16 hours in a stream of air.

50 g of alumina previously silified are impregnated in a single step, using the wet-imbibition method, with 50 cc of an aqueous solution of cobalt nitrate. This solution is obtained by dissolving 43.55 g of Co(NO₃)₂.6H₂O in such a quantity of water as to reach the above volume. The material is dried in an oven at a temperature of 120° C. for 16 hours in a stream of air and subsequently calcined at a temperature of 400° C. for 4 hours, again in a stream of air. The calcined end-product has the following chemical weight composition: 19.4 wt % of Co₃O₄, 4.0 wt % of SiO₂, complement to 100 with Al₂O₃.

The catalyst was subjected to hydrothermal treatment, as described hereunder, and to a catalytic test.

After 336 hours of hydrothermal test, the percentage of boehmite is equal to 10 wt %.

### EXAMPLE 6

### Sample F 6.5 wt % SiO₂ via MTEOS

300 g of sample A, 77.5 g of MTEOS, 300 g of technical ethanol are charged into a rotavapour flask having a volume of 2,000 ml; the reaction mixture is kept for 4 hours at 75° C. under stirring. The solvent and excess of silane are distilled under vacuum. The solid is dried for 4 hours at 140° C. and finally calcined at 400° C. for 16 hours in a stream of air.

50 g of alumina previously silified are impregnated in a single step, using the wet-imbibition method, with 50 cc of an aqueous solution of cobalt nitrate. This solution is obtained by dissolving 43.55 g of Co(NO₃)₂.6H₂O in such a quantity of water as to reach the above volume. The material is dried in an oven at a temperature of 120° C. for 16 hours in a stream of air and subsequently calcined at a temperature of 400° C. for 4 hours again in a stream of air. The calcined end-product has the following chemical weight composition: 19.4 wt % of CO₃O₄, 6.5 wt % of SiO₂, complement to 100 with Al₂O₃.

The catalyst was subjected to hydrothermal treatment, as described hereunder, and to a catalytic test.

After 672 hours of hydrothermal test, the percentage of boehmite is equal to 0 wt %.

### EXAMPLE 7

### Sample G 6.5 wt % SiO₂ via TEOS

300 g of sample A, 90.4 g of TEOS, 300 g of technical ethanol are charged into a rotavapour flask having a volume of 2,000 ml; the reaction mixture is kept for 4 hours at 75° C. under stirring. The solvent and excess of silane are distilled under vacuum. The solid is dried for 4 hours at 140° C. and finally calcined at 400° C. for 16 hours in a stream of air.

50 g of alumina previously silified are impregnated in a single step, using the wet-imbibition method, with 50 cc of an aqueous solution of cobalt nitrate. This solution is obtained by dissolving 43.55 g of Co(NO₃)₂.6H₂O in such a quantity of water as to reach the above volume. The material is dried in an oven at a temperature of 120° C. for 16 hours in a stream of air and subsequently calcined at a temperature of 400° C. for 4 hours again in a stream of air. The calcined end-product has the following chemical weight composition: 19.4 wt % of CO₃O₄, 6.5 wt % of SiO₂, complement to 100 with Al₂O₃.

The catalyst was subjected to hydrothermal treatment, as described hereunder, and to a catalytic test.

After 672 hours of hydrothermal test, the percentage of boehmite is equal to 3.5 wt %.

### General Description of the Hydrothermal Tests

In order to compare the resistance to hydration of the various catalysts, prepared according to the procedure described above, a hydrothermal test was effected suitable for simulating particularly forced Fischer-Tropsch conditions in terms of partial water pressure.

6.60 g of H₂O (0.37 mol, vol.=6.6 cm⁻³), 18.35 g of n-C₇H₁₆ (0.18 mol, vol.=26.8 cm⁻¹) and 13.20 g of n-C₅H₁₂ (0.18 mol, vol.=21.1 cm⁻³) are charged into a stainless steel autoclave having a volume of 260.0 cm⁻³; 2.00 g of sample are added to the mixture thus obtained. The autoclave is hermetically closed, placed in a rotating oven at a temperature of 200° C. for a certain time. At the end of the pre-established time, the autoclave is cooled, the solid separated from the solvent by filtration, washed with acetone and finally dried at 60° C. for 4 h. The solid is subsequently subjected to XRD analysis for the phase control.

Under the test operating conditions, the total pressure is 32 bars with the liquid/vapour composition indicated in Table 1. Under these conditions, the sample proves to be subject to a partial H₂O pressure equal to 15.5 bars corresponding to CO conversions >75% under FT reaction conditions.

TABLE 1
Hydrothermal conditions

| Molar composition | Total | Vapour phase | Liquid phase |
|---|---|---|---|
| H₂O | 0.4868 | 0.4845 | 1 |
| n-C₇H₁₆ | 0.3380 | 0.3395 | — |
| n-C₅H₁₂ | 0.1752 | 0.1760 | — |

The data relating to the hydrothermal tests indicated above for each catalyst are summarized in Table 2.

TABLE 2

| Example | Sample | Wt % SiO₂ | hours | XRD | |
|---|---|---|---|---|---|
| Example 3 | C | 0 | 120 | 20 | wt % boehmite |
| Example 4 | D | 4.0 | 336 | 14 | wt % boehmite |
| Example 5 | E | 4.0 | 336 | 10 | wt % boehmite |
| Example 6 | F | 6.5 | 672 | 0 | wt % boehmite |
| Example 7 | G | 6.5 | 672 | 3.5 | wt % boehmite |

The formation of boehmite indicates a low hydrothermal stability of the material. In particular, for contents of boehmite higher than 7-8%, the mechanical resistance properties of the catalyst are so low that they cannot be used in the reaction. Sample C therefore has an absolutely insufficient stability, whereas samples D and E have a stability which is lower than 300 hours of reaction. The use of MTEOS (sample E) instead of silica (Sample D) causes an improvement, but the result is still unsatisfactory. An increase in the content of MTEOS, on the other hand, allows a catalyst (sample F) with a high stability to be obtained: there is no presence of boehmite even after 672 hours of testing. If the same quantity of silica as alkoxide (TEOS) is added instead of MTEOS, the stability of the sample is not good: the formation process of boehmite can already be observed.

### Description of the Catalytic Tests

The samples which showed the best mechanical stability under hydrothermal conditions were subjected to a catalytic test to verify their activity in the Fischer-Tropsch synthesis.

The catalyst is charged in the pre-established quantities (20 cc) into the fixed bed tubular reactor. The activation of the catalyst is effected in situ by reduction in hydrogen (2 Nl/h/lcat) and nitrogen (1 Nl/h/lcat) at a temperature ranging from 320-450° C. and a pressure of 1 bar for 16 hours. At the end, the reactor is cooled in a stream of nitrogen.

During this phase, the system is brought to a final operating pressure of 20-30 bars. The reagent mixture consisting of H₂ and CO is introduced in a stoichiometric ratio of 2:1 by the progressive inlet of CO—H₂ and a reduction in the feeding of N₂ as indicated in Table 3:

TABLE 3
Feeding conditions in the activation phase

| | H₂ flow-rate | CO flow-rate | N₂ flow-rate |
|---|---|---|---|
| Time range (h) | (NI/h) | (NI/h) | (NI/h) |
| 0-0.5 | 10 | 30 | 200 |
| 0.5-1 | 10 | 30 | 150 |
| 1-1.5 | 10 | 30 | 100 |
| 1.5-2 | 10 | 30 | 50 |
| 2.5-3 | 10 | 30 | 0 |

At the end of said activation phase, the system proves to be completely free of gaseous diluent (nitrogen) and under the desired pressure conditions, space velocity, H₂/CO ratio. The temperature is then raised to 215° C. in about 5 h. The effluent gas from the reactor passes through a meter and a subsequent sampling system for gas-chromatographic analysis. The solid and liquid effluents are analyzed with a suitable gas-chromatographic apparatus for the total quantification. In order to normalize the catalytic activity data of the various tests, with respect to the effective cobalt content, the yield to products containing carbon (hydrocarbons and CO₂) normalized for the effective moles of cobalt present in the catalyst and for the time unit: defined as Co-TY (Cobalt-Time Yield)=converted CO moles/total Co moles/hour), is used as a comparison parameter.

### EXAMPLE 8

### Catalytic Tests

The example compares the catalytic performances of the samples having an improved hydrothermal stability, i.e. F (6.5% of SiO₂ via MTEOS) and G (6.5% SiO₂ via TEOS), evaluated with tests in a fixed bed reactor. The data of the catalytic activity tests, for the samples in question, are indicated in Table 4 and compared, with isotemperature, in terms of conversion and productivity to heavy products (C₂₂₊).

TABLE 4
Performances of catalysts F and G

| | F | G |
|---|---|---|
| GHSV (NI/I_{cat}/h) | 1500 | 1500 |
| Temperature (° C.) | 215 | 215 |
| Test pressure (abs. bars) | 21 | 21 |
| Effective H₂/CO | 2.00 | 2.00 |
| CO conversion (%) | 43.4 | 43.7 |
| Co-TY (mol conv. CO/h/mol Co) | 5.1 | 4.7 |
| C₂₊ productivity (gC₂₊/h/Kg_{cat}) | 129 | 132 |
| C₂₂₊ selectivity (weight %) | 26.1 | 27.2 |
| CH₄ selectivity (weight %) | 9.9 | 10.0 |

The results confirm that the catalysts being tested have a high specific activity for the reaction in question. In particular, a comparison between catalysts F and G, having the same silica content of 6.5 wt % and the same catalytic performances, demonstrates how the use of the MTEOS silane instead of the TEOS alkoxide gives the catalyst a better hydrothermal stability.

Samples prepared with the MTEOS silane, with a content of SiO₂ within the range of 4.5 wt % to 10 wt %, are therefore preferred.

1. A process for the preparation of a Fischer-Tropsch catalytic precursor based on cobalt supported on alumina, optionally containing up to 10% by weight of silica, which comprises:
a) treatment of alumina with a silicon compound selected from those having general formula (I)
Si(OR)₄₋ₙR′ₙ (I)
wherein n ranges from 1 to 3
wherein R′ is selected from primary hydrocarbyl radicals having from 1 to 20 carbon atoms;
wherein R is selected from primary hydrocarbyl radicals having from 1 to 6 carbon atoms;
b) drying and subsequent calcination of the modified carrier obtained at the end of step (a) thus obtaining a silanized carrier;
c) subsequent deposition of cobalt on the silanized carrier obtained at the end of step (b);
d) drying and subsequent calcination of the supported cobalt obtained at the end of step (c) thus obtaining the final catalytic precursor;
the above final catalytic precursor having a content of SiO₂ deriving from the compound having general formula (I) ranging from 4.5 to 10% by weight.

2. The process according to claim 1, wherein the alumina is gamma-alumina.

3. The process according to claim 1, wherein R′ is a primary C₁-C₁₀ alkyl radical.

4. The process according to claim 1, wherein R is a primary C₁-C₄ alkyl radical.

5. The process according to claim 1, wherein R and R′ are selected from —CH₃, —CH₂CH₃ and “n”=1.

6. The process according to claim 1, wherein step (a) takes place by treatment of the alumina with a solution, preferably ethanolic, of the compound having general formula (I).

7. The process according to claim 1, wherein the calcination of step (b) is effected at a temperature ranging from 300° C. to 500° C. for a time ranging from 2 to 20 hours in a stream of air.

8. The process according to claim 1, wherein step (c) is effected using an aqueous solution of cobalt nitrate according to the wet-imbibition technique.

9. The process according to claim 1, wherein the calcination steps (b) and (d) are effected at a temperature ranging from 300° C. to 500° C.

10. The process according to claim 1, wherein the catalytic precursor has a content of CO₃O₄ ranging from 15 to 25% by weight.

11. The process according to claim 1, wherein the catalytic precursor has a content of SiO₂ deriving from the compound having general formula (I) ranging from 4.5 wt to 10% wt.

12. The process according to claim 10, wherein the catalytic precursor has a content of SiO₂ deriving from the compound having general formula (I) ranging from 6 to 7% wt.

13. Use of the catalytic precursor according to claim 1 in Fischer-Tropsch processes.
